(19) United States
(12) Patent Application Publication
(10) Pub. No.: US 2021/0401592 A1
(43) Pub. Date: Dec. 30, 2021

(54) PROSTHESIS COMPRISING A PROGRAMMED MAGNETIC INTERLOCK

(71) Applicant: Medtronic, Inc., Minneapolis, MN (US)

(72) Inventors: Shayla O'Brien, Phoenix, AZ (US); William C. Harding, Chandler, AZ (US); Seth O'Brien, Phoenix, AZ (US)

(21) Appl. No.: 17/354,507

(22) Filed: Jun. 22, 2021

Related U.S. Application Data
(60) Provisional application No. 63/045,338, filed on Jun. 29, 2020.

(51) Int. Cl. A61F 2/70; A61F 2/66; A61F 2/80

(52) U.S. Cl. CPC A61F 2/70; A61F 2002/5083; A61F 2/80; A61F 2/66

(57) ABSTRACT

In some examples, a prosthesis system comprises a socket housing comprising a socket configured to receive a residual limb of a wearer. The socket housing further comprising a pylon extending distally of the socket. The socket housing further comprising a first interlock structure at a distal end of the pylon where the first interlock structure comprises a first one or more programmed magnets. The prosthesis system further comprising an extremity unit configured to be attached to and removed from the pylon where the extremity unit comprises a second interlock structure comprising a second one or more programmed magnets. The pylon and the extremity unit are configured such that, when the extremity unit is attached to the pylon, the first and second one or more programmed magnets are positioned and oriented to magnetically attract each other and secure the extremity unit to the pylon.

This application claims the benefit of U.S. Provisional Application Ser. No. 63/045,338, filed Jun. 29, 2020, the entire content of which is incorporated herein by reference.

### TECHNICAL FIELD

This disclosure relates to prostheses and, more particularly, techniques to attach and detach prosthetic components, allowing them to be interchanged by the wearer.

### BACKGROUND

Individuals who wear a lower limb prosthesis after amputation (hip disarticulation, transfemoral, knee disarticulation, transtibialor Syme's) all utilize prosthetic components such as knees and feet in one way or another. Similarly, individuals who wear an upper extremity prosthesis after amputation (shoulder disarticulation, transhumeral, elbow disarticulation, transradial or wrist disarticulation) all utilize prosthetic components such as elbows, hooks or hands in some manner. A number of factors may be considered in the design of prosthetic components, such as: (1) fit—athletic/active amputees, or those with bony residua, may require a carefully detailed socket fit, while less-active amputees may be comfortable with a ‘total contact’ fit and gel liner; (2) energy storage and return storage of energy acquired through ground contact and utilization of stored energy for propulsion; (3) energy absorption minimizing the effect of high impact on the musculoskeletal system; (4) ground compliance stability independent of terrain type and angle; (5) rotation ease of changing direction; (6) weight maximizing comfort, balance and speed; and (7) suspension how the socket will join and fit to the limb. It is very common for active amputees to change components for different activities such as running, cycling, snowboarding, water sports, day-to-day use, etc.

Amputees deal with a cumbersome situation when needing to swap out one component for another. Conventional coupling systems for prostheses require one to disassemble one component from a pylon coupled to the socket and reassemble another component on the pylon. This process typically needs to be completed by a skilled professional, such as a prosthetist, to ensure proper alignment. There is evidence to suggest a causal relationship between a misaligned prosthesis and a wearer's limited mobility, increased joint pain, increased risk of balance related falls, increased risk of limb infection and overall reduced quality of life. Thus, for wearers having multiple activity specific prosthetic components, conventional prosthesis attachment requires a skilled professional, but the associated burden (e.g., cost, time, etc.) is impractical and limiting.

### SUMMARY

In general, examples of the present disclosure describe a prosthesis system with a programmed magnetic interlock for selectively attaching and detaching a socket and an extremity unit. In some examples, the programmed magnetic interlock is a twist and release interlock. The programmed magnetic interlock provides a magnetic attraction force until a socket housing and an extremity unit are at a predetermined angle to one another. The predetermined angle may also be the angle for attachment of the socket housing to the extremity unit. A prosthesis wearer may then couple the magnetic interlocks and rotate laterally the extremity unit from the predetermined angle to a use position where the programmed magnets have their greatest magnetic attraction. The prosthesis may include a three-dimensional key to assist in prevention of accidental release.

In some examples, this disclosure describes a prosthesis system comprising a socket housing comprising a socket configured to receive a residual limb of a wearer. The socket housing further comprising a pylon extending distally of the socket. The socket housing further comprising a first interlock structure at a distal end of the pylon where the first interlock structure comprises a first one or more programmed magnets. The prosthesis system further comprising an extremity unit configured to be attached to and removed from the pylon where the extremity unit comprises a second interlock structure comprising a second one or more programmed magnets. The pylon and the extremity unit are configured such that, when the extremity unit is attached to the pylon, the first and second one or more programmed magnets are positioned and oriented to magnetically attract each other and secure the extremity unit to the pylon.

In some examples, this disclosure describes a method of manufacturing a prosthesis system comprising forming a socket housing configured to receive a residual limb of a wearer. The socket housing comprising a socket configured to receive a residual limb of a wearer. The socket housing further comprising a pylon extending distally of the socket and a first interlock structure at a distal end of the pylon wherein the first interlock structure comprises a first one or more programmed magnets. The method further comprising forming an extremity unit configured to be attached to and removed from the pylon where the extremity unit comprises a second interlock structure comprising a second one or more programmed magnets. The pylon and the extremity unit are configured such that, when the extremity unit is attached to the pylon, the first and second one or more programmed magnets are positioned and oriented to magnetically attract each other and secure the extremity unit to the pylon.

In some examples, this disclosure describes a prosthesis system comprising a socket housing comprising a socket configured to receive a residual limb of a wearer. The socket housing further comprising a pylon extending distally of the socket and a first interlock structure at a distal end of the pylon. The first interlock structure comprising one or more programmed magnets and a three-dimensional key comprising a cylinder extending longitudinally away from the pylon. The cylinder with at least one cam extending outward transversely from the longitudinal axis. The prosthesis system further comprising an extremity unit configured to be attached to and removed from the pylon where the extremity unit comprises a second interlock structure comprising one or more programmed magnets. The extremity unit further comprising a cylindrical channel that comprises at least one slot that is configured to receive the at least one cam portion. The pylon and the extremity unit are configured such that, when the extremity unit is attached to the pylon, the one or more programmed magnets of the first interlock structure and the second interlock structure are positioned and oriented to magnetically attract each other and secure the extremity unit to the pylon. The first and second one or more programmed magnets are configured such that movement of the extremity unit in a predetermined direction relative to the pylon releases the magnetic attraction of the first and second one or more programmed magnets to each other and the first and second one or more programmed magnets comprise twist release programmed magnets, and the predetermined direction comprises rotation of the extremity unit about a longitudinal axis of the pylon. The first one or more programmed magnets and the second one or more programmed magnets are programed to have a greater magnetic pull force when the first one or more programmed magnets and the second one or more programmed magnets are positioned so the pylon and foot are properly aligned for use by a wearer.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a conceptual diagram of an example lower-extremity prosthesis system including an interlocking magnetic structure.

FIG. 1B is a conceptual diagram of an example configuration of a slot within an interlock structure according to the present disclosure.

FIG. 2A is a conceptual diagram of an example of magnetic flux lines emanating from a first interlock structure according to the present disclosure.

FIG. 2B is a conceptual diagram of an example of magnetic flux lines emanating from a second interlock structure according to the present disclosure.

FIG. 3A is a conceptual diagram of a full repulsion state for a first and second interlock structure according to the present disclosure.

FIG. 3B is a conceptual diagram of a full attraction state for a first and second interlock structure according to the present disclosure.

FIG. 4 is a flow diagram of an example method of manufacturing a prosthesis system in accordance with examples of the present disclosure.

FIG. 5 is a flow diagram of an example method of use for a prosthesis system in accordance with examples of the present disclosure.

### DETAILED DESCRIPTION

In general, examples of the present disclosure describe a prosthesis system with a twist and release programmed magnetic interlock. The programmed magnetic interlock may be configured with a first interlock structure extending from a socket housing. The first interlock structure may comprise one or more programmed magnets. A second interlock structure may be configured to accept the first interlock structure and may be coupled to an extremity unit and comprise one or more programmed magnets. In some examples, the first and/or second interlock structures may be entirely comprised of programmed magnets. In some examples, the first and/or second interlock structures may be partially comprised of programmed magnets, e.g., programmed magnets may be placed or formed at selected locations of the interlock structure.

Although described primarily in the context of examples in which an interlock structure extending from a socket housing is configured to be mechanically received by an interlock structure of an extremity unit, the techniques described herein are not limited to such examples. In some examples, an interlock structure may extend from an extremity unit and be received by an interlock structure of a socket housing. In some examples, interlock structures of both units may include portions that extend to be received by the interlock structure of the other unit. Any structure(s) configured to mechanically interlock two units to each other may be configured with one or more programmed magnetics according to the techniques of this disclosure. Furthermore, in some examples, programmed magnets lock the units together in the absence of any mechanical interlock.

A wearer of the extremity unit may couple the first interlock structure with the second interlock structure at a first predetermined angle (e.g., 90° from a use or “locked” position about a longitudinal axis). This may be the angle of attachment and/or detachment and also the angle with the maximal amount of magnetic repulsion between the first interlock structure programmed magnets and the second interlock structure programmed magnets. The wearer may rotate the extremity unit to a second predetermined angle, which correlates with an extremity unit “use” position. As the angle between the first interlock programmed magnets and the second interlock programmed magnets changes, the magnetism changes as well from repulsion to attraction where the magnetic attraction is maximal between the first interlock structure programmed magnets and the second interlock structure programmed magnets when in the “use” position.

The magnetic attraction may lock the extremity unit in place with a strong magnetic force. In some examples, the programmed magnets may be configured to allow the rotation, e.g., about the longitudinal axis, to release the interlock, but may strongly resist other types of relative motion, such as pulling the units apart longitudinally or transversely, e.g., with a shearing motion.

As a support feature for patient safety and to reduce the risk of an accidental release of the extremity unit, the prosthesis system may include a three-dimensional key interlocking with a channel to assist in prevention of accidental release. In some examples, this three-dimensional key may have cams as part of the first interlock structure. The cams are configured to be received by slots within a channel formed by the second interlock structure. In examples of the present disclosure, the cams and the slots may be entirely comprised of programmed magnets. In some examples, the cams and the slots may be partially comprised of programmed magnets. In other examples, only the cams or only the slots are comprised of programmed magnets.

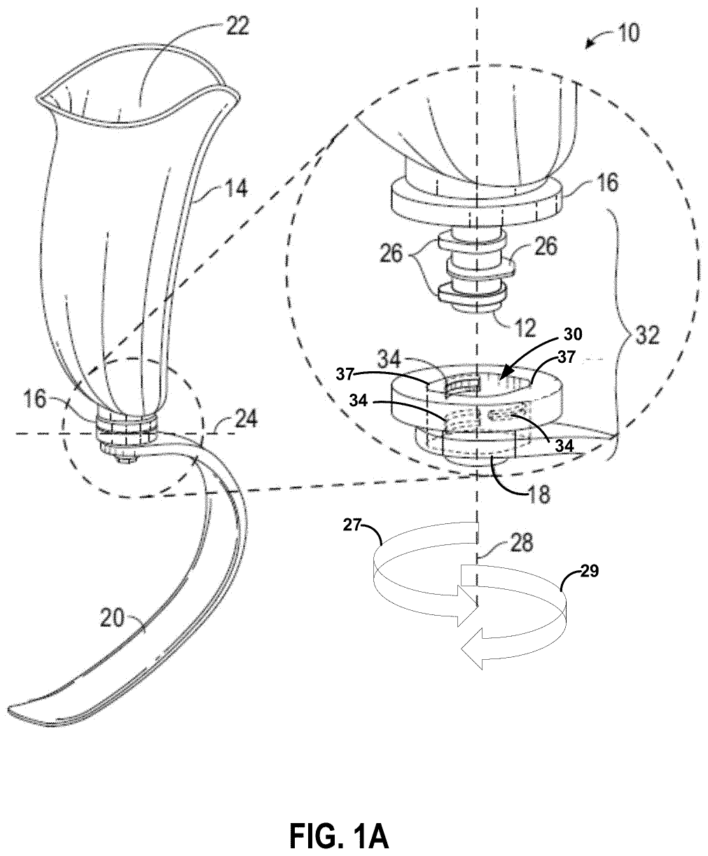

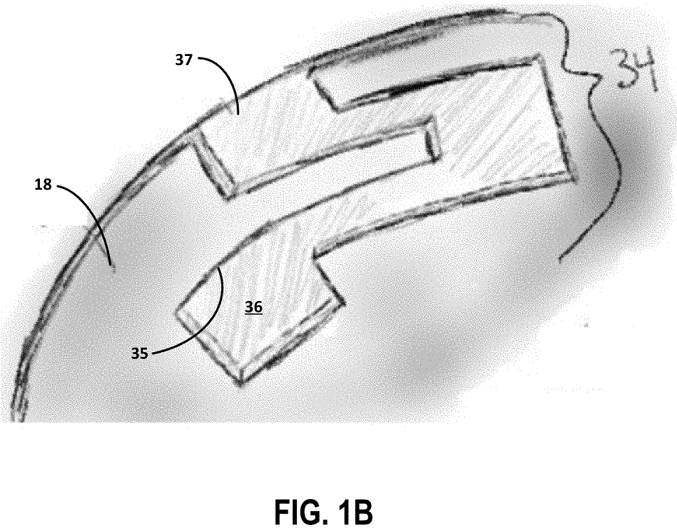

FIG. 1A is a conceptual diagram of an example lower-extremity prosthesis system 10 including interlocking magnetic structure 32. While the example prosthesis system 10 illustrated in FIG. 1A is a lower-extremity prosthesis system 10, the examples and techniques of the present disclosure may be applied to an upper-extremity prosthesis system as well. Prosthesis system 10 may include any prosthetic device, such as a hand, foot, lower leg, lower arm, leg, arm, ear, finger(s), or toe(s), as the examples of the present disclosure may be extended to any part of the human body which may be substituted with a prosthesis.

In the example illustrated by FIG. 1A, lower-extremity prosthesis system 10 (hereinafter “prosthesis system 10”) includes a socket housing 14 that includes a socket 22 configured to receive a residual limb of a wearer (not shown in FIG. 1A). Pylon 16 may extend distally of socket 22. A first interlock structure 12 may be located at or near a distal end of pylon 16. Prosthesis system 10 further comprises an extremity unit 20 configured to be attached to and removed from pylon 16. Extremity unit 20 may have a second interlock structure 18. Pylon 16 and extremity unit 20 are configured such that, when extremity unit 20 is attached to pylon 16, first interlock structure 12 and second interlock structure 18 are positioned and oriented to secure extremity unit 20 to pylon 16.

Prosthesis system 10 may be an artificial device that replaces a missing body part, which may be lost through trauma, disease, or a condition present at birth (e.g., congenital disorder). Prosthesis system 10 may be intended to restore the normal functions of the missing body part. Amputee rehabilitation is primarily coordinated by a physiatrist as part of an inter-disciplinary team consisting of physiatrists, prosthetists, nurses, physical therapists, and occupational therapists. Prosthesis system 10 may be created by hand or with Computer-Aided Design (CAD) software. The two main subcategories of lower extremity prosthesis systems 10 are transtibial (any amputation transecting the tibia hone or a congenital anomaly resulting in a tibial deficiency), and transfemoral (any amputation transecting the femur bone or a congenital anomaly resulting in a femoral deficiency). In the prosthesis industry, a transtibial prosthesis leg is often referred to as a “BK” or below-the-knee prosthesis while the transfemoral prosthesis leg is often referred to as an “AK” or above-the-knee prosthesis. Other, less prevalent lower extremity cases include the following: hip disarticulations usually refer to when an amputee or congenitally challenged wearer has either an amputation or anomaly at or in close proximity to the hip joint. Knee disarticulations usually refers to an amputation through the knee disarticulating the femur from the tibia. Symes is an ankle disarticulation while preserving the heel pad.

First interlock structure 12 and second interlock structure 18 may have programmed magnets (not shown in FIG. 1A) respectively that incorporate correlated patterns of magnets with alternating polarity, designed to achieve a desired behavior and deliver stronger local force. By varying the magnetic fields and strengths, different mechanical behaviors may be controlled.

Correlated magnet pairs may be programmed to attract or repel with a prescribed force and engagement distance, or, to attract or repel at a certain spatial orientation (discussed in greater detail below). Correlated magnets may he programmed to interact only with other magnetic structures that have been coded to respond. Correlated magnets may even be programmed to attract and repel at the same time. Compared to conventional magnets, the correlated magnet provides a much stronger holding force to the target and stronger shear resistance. The programmed behavior is achieved by creating multipole structures comprising multiple magnetic elements (e.g., magnetic pixels; maxels) of varying size, location, orientation, and saturation. In some examples, the sizes of maxels range from 1 mm to 4 mm. By overlapping these maxels, a very intricate magnetic field may be produced. There are four main functions that correlated magnets may achieve: align, attach, latch, and spring.

Programmed magnets may be programmed, or coded, by varying the polarity and/or field strengths of each source of the arrays of magnetic sources that make up each structure. The resulting magnetic structures may be one-dimensional, two-dimensional, three-dimensional, and even four-dimensional if produced using an electromagnetic array. Correlated magnetic structures may be developed from ferrites, rare-earth materials (e.g. Neodymium magnet, Samarium-cobalt magnet), ceramics, and electromagnets alike, and the correlation effects are scalable from very large permanent magnets to nanometer-scale devices. Multipole magnetic devices may be constructed from discrete permanent magnets, or by exposing heated magnetizable material to a coded magnetic field.

When programmed magnets of the first interlocking structure 12 are in sufficient proximity and at a predefined orientation relative to programmed magnets of the second interlock structure 18, the programmed magnets simulate the functionality of a lock with forces resisting changes from a predefined orientation between first interlock structure 12 and second interlock structure 18. In this manner, first interlock structure programmed magnets and second interlock structure programmed magnets act as an attachment mechanism between first interlock structure 12 and second interlock structure 18, resisting axial movement along vertical axis 28 and along horizontal axis 24. Further, first interlock structure 12 may have optional cams 26 and second interlock structure 18 may have optional slots 34 which may accept cams 26 and, in this manner, cams 26 and slots 34 provide additional locking strength to lock extremity unit 20 in place and act to reduce the risk of an accidental release.

Socket housing 14 may be adapted for a residual arm or leg. Socket housing 14 may be made from hard epoxy or carbon fiber. Socket housing 14 or other “interfaces” may be made more comfortable by lining them with a softer, compressible foam material that provides padding for the bone prominences. A self-suspending or supra-condylar socket design is useful for those with short to mid-range below elbow absence. Longer limbs may require the use of a locking roll-on type inner liner or more complex harnessing to help augment suspension. Socket housing 14 serves as an interface between the residuum (the remaining portion of an amputee's residual limb, not shown in FIG. 1A) and prosthesis extremity unit 20, ideally allowing comfortable weight-bearing, movement control and proprioception.

Shank or pylon 16 creates distance and support between a knee-joint (not shown in FIG. 1A) and extremity unit 20 (in case of an upper-leg prosthesis) or between socket housing 14 and extremity unit 20. The type of connectors that are used between pylon 16 and the knee/foot determines whether prosthesis 10 is modular or not. Modular means that the angle and the displacement of extremity unit 20 in respect to socket 14 may be changed after fitting,

Providing contact to the ground, extremity unit 20 may provide shock absorption and stability during stance. Additionally, extremity unit 20 may influence gait biomechanics by its shape and stiffness. This is because the trajectory of the center of pressure (COP) and the angle of the ground reaction forces is determined by the shape and stiffness of extremity unit 20 and needs to match the subject's build in order to produce a normal gait pattern. Evidence comparing different types of extremity units 20 and ankle prosthesis devices is not strong enough to determine if one mechanism of anklel/foot is superior to another. In any case, in convention prosthesis systems, a wearer may need to disassemble the extremity unit from the pylon, a process requiring a skilled professional to ensure proper alignment. Further, there is a causal relationship between a misaligned extremity unit and a wearer's limited mobility, increased joint pain, increased risk of balance related falls, increased risk of limb infection and overall reduced quality of life.

Prosthesis system 10 may be an example of an artificially replaced limb located at the hip level or lower. Lower-extremity amputations may be estimated worldwide between 2.0-5.9 per 10,000 people. Estimates of birth prevalence rates of congenital limb deficiency range between 3.5-7.1 cases per 10,000 births. The two main subcategories of lower extremity prosthesis devices may be transtibial (e.g., any amputation transecting the tibia bone or a congenital anomaly resulting in a tibial deficiency), and transfemoral (e.g., any amputation transecting the femur bone or a congenital anomaly resulting in a femoral deficiency). In the prosthetic industry, a transtibial prosthesis leg may be often referred to as a “BK” or below-the-knee prosthesis, while the transfemoral prosthesis leg may be often referred to as an “AK” or above-the-knee prosthesis.

Other, less prevalent lower extremity cases include the following: (1) hip disarticulations, e.g., when an amputee or congenitally challenged wearer has either an amputation or anomaly at or in close proximity to the hip joint; (2) knee disarticulations, e.g., an amputation through the knee disarticulating the femur from the tibia; and (3) Symes, which may be an ankle disarticulation while preserving the heel pad.

Amputees may deal with cumbersome situations when wanting to swap out a prosthetic component, such as a foot. It is common for amputees to have several different prosthetic feet for running, cycling, snowboarding, etc. Therefore, many amputees have entirely different prostheses for different activities. This may be very expensive as lower limb prostheses may range between $3,500 and $50,000. The swapping out of entire prostheses is costly, burdensome and impractical.

Prosthesis system 10 according to the examples of this disclosure enhances the abilities of a wearer to not only move and function normally, but also to perform extremity unit 20 swapping without the need for a professional. Prosthesis system 10 addresses issues associated with wearer comfort caused by a misaligned prosthesis and allows for increased physical activity. Examples of the present disclosure increase ease of use for prosthesis system 10 including the extremity unit 20 swap out and increases in the ability for the prosthesis system wearer to be more physically active.

Prosthesis system 10 allows a wearer to twist extremity unit 20 to a predetermined angle (e.g., a 90° angle from the use orientation) to remove extremity unit 20. Examples of the present disclosure discuss a “twist and release” prosthesis system 10 where extremity unit 20 may be twisted and released from pylon 16. An amputee may rotate extremity unit 20 (e.g., a lateral rotation about axis 28) to disconnect extremity unit 20 from pylon 16. The same or a separate extremity unit 20 may then be coupled to pylon 16 by coupling and rotating extremity unit 20 into a use position. The programmable magnets have their strongest attraction at a location providing proper alignment (e.g., proper for wearer use; the “use” position) for the wearer. The magnets may be programmed and placed on the units based on evaluation of what is the proper use position for a given user. Thus, extremity unit 20 is held in place by a magnetic attraction between programmed magnets.

To assist in reducing the risk of accidental release of extremity unit 20 (e.g., a situation where extremity unit 20 is twisted during a sharp turn or sport activity), first 12 and second interlock structures 18 may have a three-dimensional “key” design 32, utilizing cams 26 for first interlock structure 12 and slots 34 for second interlock structure 18. Each cam 26 of first interlock structure 12 may be received within opening 30 and after insertion, extremity unit 20 is rotated (about arrow 29) and each of cams 26 may be received within a correlating slot 34 of second interlock structure 18. Once within opening 30 and after the wearer rotates extremity unit 20 into a use position, cams 26 are rotated into slots 34. Cam members 26 may not be necessary and in some examples, the magnetic force between programmed magnets may be relied upon to securely hold extremity unit 20 to pylon 16. However, in some examples where the wearer participates in physically demanding activities, such as triathlons or mountain climbing, then the wearer may desire an additional barrier to extremity unit 20 accidentally separating and choose to utilize cam members 26 and slots 34. In other examples, first interlock structure 12 may couple with second interlock structure 18 with a press and release cam or tab member which latches into a slot. In other examples a pin and slot may be used where the user can slide a pin through a slot, similar to a cotter pin, when the extremity unit is properly placed. In other examples, a ball and hitch may be used to couple the pylon with the extremity unit. In yet another example, a first magnet may be coupled to first interlock structure 12 at the end of first interlock structure 12 (e.g., where the reference line to element 12 is pointing in FIG. 1A) and a second magnet capable of attraction to the first magnet may be located at the end of second interlock structure 18 at the base (e.g., where the reference line is pointing to element 18 in FIG. 1A) to prevent separation of the foot or other component during intensive activity.

Opening 30 is shown with a wider opening at location 37. The wide opening at location 37 allows for the receipt of cams 26. Thus, first interlock structure 12 may be received by second interlock structure 18 and then when second interlock structure 18 is turned to magnetically secure extremity unit 20 into the use position, cams 26 are also held within slots 34 and thus resistive to extremity unit 20 being pulled back through opening 30.

In an example of the present disclosure prosthesis system 10 may comprise socket housing 14 where socket 22 may receive a residual limb of a wearer (not shown in FIG. 1A). Pylon 16 may extend distally from socket 22 and first interlock structure 12 at a distal end of pylon 16. First interlock structure 12 may have one or more programmed magnets (not shown if FIG. 1A). In some examples, first interlock structure 12 may be comprised entirely of programmed magnets. In other examples, portions of first interlock structure 12 may be comprised of programmed magnets. In some examples, the portion(s) of first interlock structure 12 comprised of or including programmed magnets may correlate with the location of one or more cams 26, and portion(s) of second interlock structure 18 comprised of or including programmed magnets may correlate with the location of one or more slots 34. In some examples, the portion(s) of first interlock structure 12 comprised of or including programmed magnets may be a distal portion of pylon and/or a distal portion of first interlock structure 12, and the portion(s) of second interlock structure 18 comprised of or including programmed magnets may be a proximal portion of second interlock structure 18 and/or a distal end of opening 30.

Prosthesis system 10 may comprise extremity unit 20 that may be attached to and removed from pylon 16. Extremity unit 20 may comprise second interlock structure 18 comprising a second one or more programmed magnets (not shown in FIG. 1A). Pylon 16 and extremity unit 20 may be coupled when extremity unit 20 is attached to pylon 16 and the first and second one or more programmed magnets are positioned and oriented to magnetically attract each other and secure extremity unit 20 to pylon 16.

FIG. 1B is a conceptual diagram of an example configuration of slot 34 within an interlock structure 18 according to the present disclosure. Slot 34 may be formed in the surface of interlock structure 18 that defines opening 30, and may include the wider portion 37 of opening into 30 which cam 26 is inserted. As illustrated by the example of FIG. 1B, once cam 26 is inserted longitudinally into slot 34 via wider portion 37, attachment of extremity unit 20 to pylon 16 may require one or more additional rotational and/or longitudinal movements of extremity unit 20 relative to pylon 16 until extremity unit 20 reaches its use position and is securely locked to pylon 16. In the example illustrated by FIG. 1B, the user may move extremity unit longitudinally toward pylon 16 a first time, rotationally in a first direction, longitudinally toward pylon 16 a second time, rotationally in a second direction, which may be opposite the first direction, and longitudinally toward pylon 16 a third time. At this time, cam 26 is seated in lock location 36 and a simple longitudinal separation of extremity unit 20 from pylon 16 is prevented by slot wall 35. In this manner, slot 34 may provide a three-dimensional key 32. A user may separate extremity unit 20 from pylon 16 by reversing the series of movements required for attachment, and thus moving cam 26 back through slot 34.

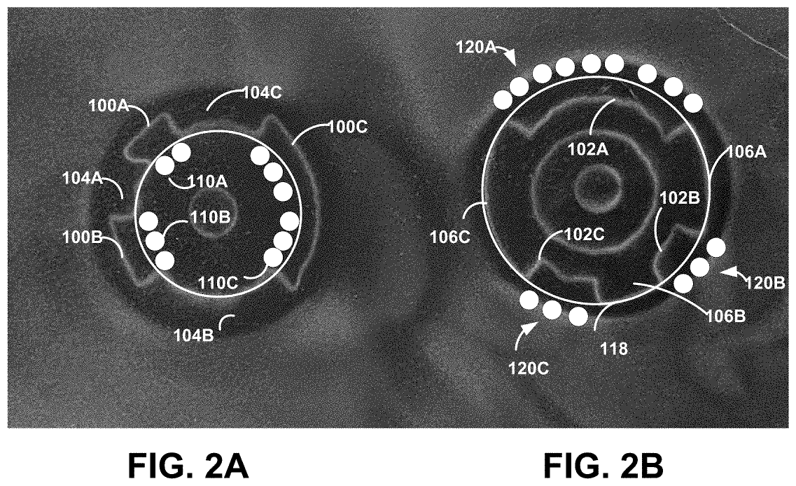

FIGS. 2A and 2B are conceptual diagrams of an example representation of magnetic flux lines emanating from programmed magnets, e.g., of a first interlock structure and a second interlock structure according to the present disclosure.

In FIG. 2A, a plurality of magnetic flux lines 100A, 100B, and 100C (hereinafter referred to as “magnetic flux lines 100”) extend from first programmed magnets 110A, 110B, and 110C (hereinafter referred to as “first programmable magnets 110”) of a first interlock structure 12.

In FIG. 2B, magnetic flux lines 102A, 102B, and 102C (hereinafter referred to as “magnetic flux lines 102”) emanate from second programmed magnets 120A, 120B, and 120C (hereinafter referred to as “second programmed magnets 120”), e.g., of second interlock structure 18. In one example, magnetic flux lines 100 may extend outward and correlate with cams 26 (e.g., in the example of cams 26 being part of first interlock structure 12) of first interlock structure 12. Magnetic flux lines 102 may extend inward from channel wall 118 of second interlock structure 18. In some examples, magnetic flux lines 102 may correlate with slots 34 (e.g., in the example of slots being part of the second interlock structure) of second interlock structure 18 which accept cams 26 from first interlock structure 12 in three-dimensional lock 32 (FIG. 1A). As discussed above, slots 34 may interact with cams 26 to provide a three-dimension key 32 (FIG. 1A) and provide an additional physical barrier to the magnetic barrier to further lower the risk of accidental removal of extremity unit 20.

Magnetic flux lines 100 may all have the same polarity and magnetic intensity or differing polarities and differing magnetic intensities. In some examples, areas 104A, 104B and 104C may have an opposite polarity of magnetic flux lines 100 and may have a lower magnetic intensity. In other examples, the areas of 104A, 104B and 104C have no programmed magnets and no magnetic flux lines.

Magnetic flux lines 102 may all have the same polarity, same magnetic intensity or differing polarities and differing magnetic intensities. Further, magnetic flux lines 102 may all have the same polarity, same magnetic intensity or differing polarities and differing magnetic intensities as magnetic flux lines 100 (i.e., within the desired programming results to ensure maximum magnetic attraction at a “use” position and maximum magnetic repulsion at a “detachment” position). In some examples, areas 106A, 106B and 106C may have an opposite polarity of magnetic flux lines 102 and may have a lower magnetic intensity. In other examples, the areas of 106A, 106B and 106C have no programmed magnets and no magnetic flux lines. Each of first interlock structure 12 and second interlock structure 18 may be magnetically printed and programmed as discussed above and in further detail below.

Magnetic flux lines 100 and 102 will align to provide a greatest magnetic attraction when extremity unit 20 and pylon 16 are in alignment (e.g., in a use position) which provides no misalignment, or substantially minimal misalignment, between extremity unit 20 and pylon 16. Thus, first 12 and second interlock structures 18 are designed, programmed and formed to provide an alignment that prevents limited mobility, increased joint pain, increased loss of balance and possible limb infection. Further, when a wearer decides to remove or swap out extremity units 20, they may move or rotate extremity unit 20 along axis 28 (e.g., rotated along arrow 27 (FIG. 1A). This rotation will begin to move extremity unit 20 along axis 28. As the rotation starts magnetic flux lines 100 and 102 will exit a state with the maximum magnetic attraction and a weak repulsion force begins to act on first 12 and second interlock structure 18 as magnetic flux lines 100 and 102 begin to interact at angles causing magnetic repulsion. As the prosthesis wearer rotates extremity unit 20 further along axis 28, in the direction of arrow 27, out of alignment, the repulsion force grows until the prosthesis wearer rotates extremity unit 20 to a predetermined angle that may be a detachment angle (e.g., 90° degrees to the right or left) where the repulsion magnetic force is at a maximum and extremity unit 20 may be removed from pylon 16.

Should extremity unit 20 be rotated out of place for a reason other than removal of extremity unit 20, such as a quick turn in a sporting event or a loss of balance and a turned leg, the magnetic attraction force will pull extremity unit 20 back to the fully aligned state (e.g., the state of maximal magnetic attraction for use). Further, if a part of first interlock structure 12 and second interlock structure 18, three-dimensional key 32 will assist in preventing extremity unit 20 from decoupling with pylon 16 through a physical force.

The magnetic attraction force is greatest when extremity unit 20 is properly aligned and the least when extremity unit 20 is in a removal position at a detachment angle. The magnetic attraction force may gradually decrease from the state of maximum attraction to the state of least attraction. Furthermore, the repulsion force is the greatest at the detachment angle of the extremity unit 20 and the weakest when extremity unit 20 is properly aligned for use (e.g., the use position).

A mathematical curve representing both the magnetic attraction and magnetic repulsion may be linear from maximum attraction and maximum repulsion to the weakest attraction and weakest repulsion. In another example, the curve for magnetic attraction and/or repulsion may not be linear. In another example, the magnetic repulsion force may not begin until extremity unit 20 is more than 80° out of alignment. If prosthesis system 10 is used by a very active person who participates in physically demanding sports, it may be preferred to have the repulsion force not begin until a larger angle of misalignment as an active person may have several events when misalignment may occur frequently and the magnetic attraction pulls extremity unit 20 back into alignment quickly.

In another example, the magnetic and repulsive forces may be programmed based upon a wearer's weight and strength. For example, if a wearer is a larger person (e.g., over 200 lbs.) then it may be desirable to have stronger magnetic forces in which to keep extremity unit 20 properly aligned. In another example, if extremity unit 20 were to be used by an older and lighter person, it may be desirable for the magnetic attraction forces to be lesser so the wearer may not have an overly difficult time removing extremity unit 20 from pylon 16.

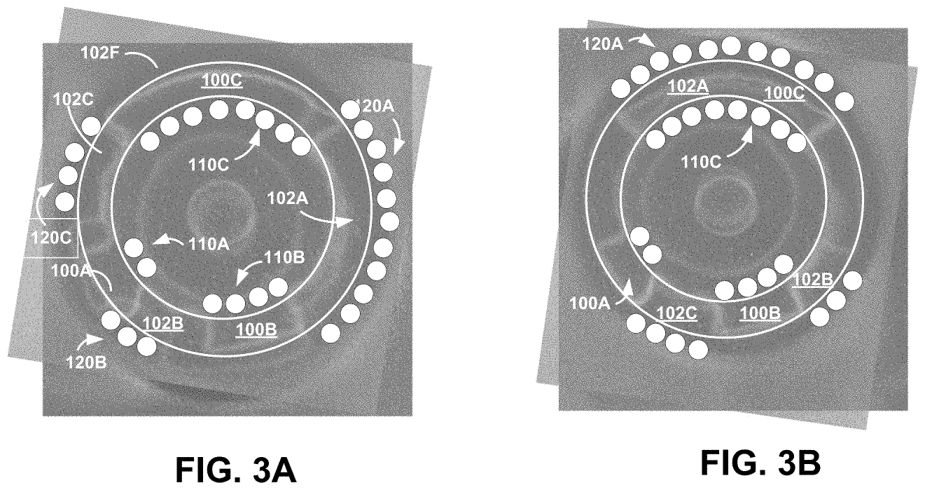

FIG. 3A is a conceptual diagram of a full repulsion state for a first 12 and second interlock structure 18 according to the present disclosure.

FIG. 3A shows a detachment position and/or a position of full magnetic repulsion between first 12 and second interlock structure 18. In one example, magnetic flux lines 100 and flux lines 102 are at an almost 90° angle to one another. In this position, programmed magnets 110 and 120 are also at an almost 90° angle to one another. In this arrangement, programmed magnets 110 and 120 are not aligned and are programmed to have a maximum magnetic repulsion force.

In the orientation of FIG. 3A, extremity unit 20 may be removed from pylon 16 relatively easily as programmed magnets 110 of first interlock structure 12 and the programmed magnets 120 of second interlock structure 18 are magnetically repelling each other. In another example, if magnetic flux lines 100 align with cams 26 and magnetic flux lines 102 align with slots 34, then as shown in FIG. 3A extremity unit 20 may be removed easily by the maximum magnetic repulsion and cams 26 being aligned with slots 34 of channel 118 (e.g., there is a maximal magnetic repulsion force and no physical barrier; cams 26 and slots 34, holding first interlock structure 12 to second interlock structure 18).

FIG. 3B is a conceptual diagram of a full attraction state for a first 12 and second interlock structures 18 according to the present disclosure.

FIG. 3B shows a use position and/or a position of full magnetic attraction between first 12 and second interlock structures 18. Programmed magnets 120 are now substantially aligned with programmed magnets 110, especially programmed magnets 120 and 110B.

In this orientation and angle of magnetic flux lines, the greatest magnitude of magnetic attraction is generated. Further, cams 26 may lay within slots 34 of channel wall 118 and extremity unit 20 and pylon 16 are coupled by magnetic and a physical connection (e.g., they may not be pulled apart).

As programmed magnets 120 and programmed magnets 110 turn from a state of full attraction (e.g., as shown in FIG. 3B) one of full repulsion (e.g., as shown in FIG. 3A) programmed magnets 120 and 110 change their properties from either repulsion or attraction. Programmed magnets 110 and 120 are physically moving during a rotational turn by the user. As programmed magnets 110 and 120 change position relative to each other, their magnetic properties change at given distances or relative positions. This programmed feature is something that may be specifically designed in the fabrication of each magnet. Once interlock structures 12 and 18 reach a point of full attraction (e.g., FIG. 3B) interlock structures 12 and 18 are locked in place (e.g., in the use position). This creates both a magnetic and physical lock; the wearer must turn and release extremity unit 20 in order to disengage the lock.

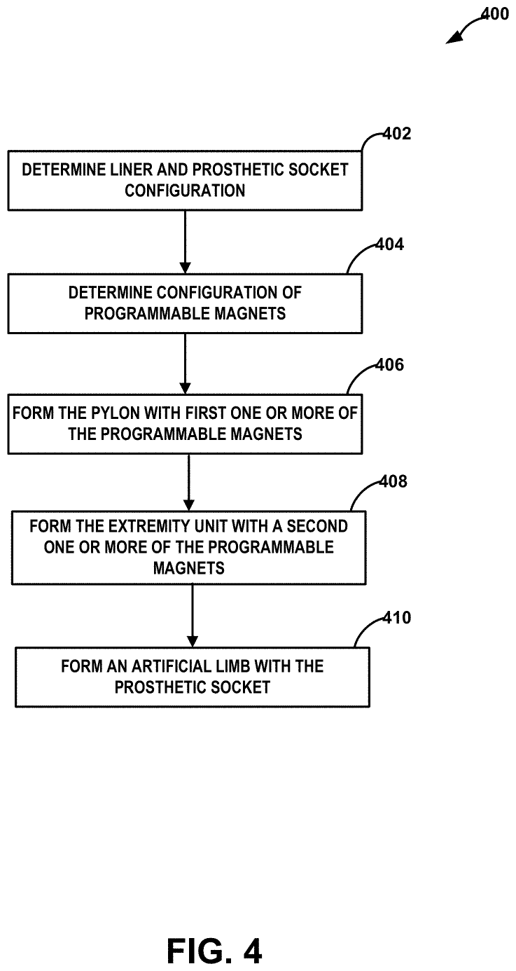

FIG. 4 is a flow diagram of an example method of manufacturing a prosthesis system 400 in accordance with examples of the present disclosure. A method of manufacturing a prosthesis 400 may have the following processes performed in any manner unless specifically stated otherwise. Prosthesis limbs may not be mass-produced to be sold in stores. Similar to the way dentures or eyeglasses may be procured, prosthesis limbs may first be prescribed by a medical doctor, usually after consultation with the amputee, a prosthetist, and a physical therapist. The patient then visits the prosthetist to be fitted with a limb. Although some parts—the socket, for instance—may be custom-made, many parts (e.g., feet, pylons) may be manufactured in a factory, sent to the prosthetist, and assembled at the prosthetist's facility in accordance with the patient's needs. At a few facilities, the limbs may be custom made from start to finish.

Accuracy and attention to detail may be important in the manufacture of prosthesis limbs, because the goal may be to have a limb that comes as close as possible to being as comfortable and useful as a natural one. Before work on the fabrication of the limb may be begun, the prosthetist evaluates the amputee and takes an impression or digital reading of the residual limb. The prosthetist then measures the lengths of relevant body segments and determines the location of bones and tendons in the remaining part of the limb. Using the impression and the measurements, the prosthetist then makes a positive model—an exact duplicate—of the residual limb. This may be most commonly made of plaster of Paris, because it dries fast and yields a detailed impression.

A sheet of clear thermoplastic may be heated in a large oven and then vacuum-formed around the positive mold. In this process, the heated sheet may be simply laid over the top of the mold in a vacuum chamber. If necessary, the sheet may be heated again. Then, the air between the sheet and the mold may be sucked out of the chamber, collapsing the sheet around the mold and forcing it into the exact shape of the mold. This thermoplastic sheet may now be the test socket; it may be transparent so that the prosthetist may check the fit.

The prosthetist works with the patient to ensure that the test socket fits properly. In the case of a missing leg, the patient walks while wearing the test socket, and the prosthetist studies the gait. The patient may be asked to explain how the fit feels; comfort comes first. The test socket may be adjusted according to patient input and retried. Because the material from which the test socket may be made may be thermoplastic, it may be reheated to make minor adjustments in shape.

A permanent socket may then be formed. Since it may be made of polypropylene, it may be vacuum formed over a mold in the same way as the test socket. It may be common for the stump to shrink after surgery, stabilizing approximately a year later. Thus, the socket may be usually replaced at that time, and thereafter when anatomical changes necessitate a change.

Plastic pieces—including soft-foam pieces used as liners or padding—may be made in the usual plastic forming methods (502). These include vacuum-forming, injection molding—forcing molten plastic into a mold and letting it cool—and extruding, in which the plastic may be pulled through a shaped die.

A plurality of programmed magnets may be programed and created (504). The programmed magnets may be formed in most any method including 3D printing and may consider factors such as the wearer's (user's) weight to properly program the desired magnetic repulsion and attraction. The programmed magnets incorporate correlated patterns of magnets with specifically programmed polarity, designed to achieve the behavior described above in FIGS. 2 and 3 and deliver stronger local force. By varying the magnetic fields and strengths, different mechanical behaviors may be controlled.

Programmed magnets may be programmed to attract or repel with a prescribed force and engagement distance, or, to attract or repel at a certain spatial orientation. Programmed magnets may be programmed to interact only with other magnetic structures that have been coded to respond, Programmed magnets may even be programmed to attract and repel at the same time. Compared to conventional magnets, programmed magnets provide stronger holding force to the target and stronger shear resistance. The programmed behavior may be achieved by creating multipole structures comprising multiple magnetic elements of varying size, location, orientation, and saturation. The sizes of these magnetic elements may range from 1 mm to 4 mm. By overlapping these magnetic elements, a very intricate magnetic field may be produced.

Programmed magnets may be programmed, or coded, by varying the polarity and/or field strengths of each source of the arrays of magnetic sources that make up each structure. The resulting magnetic structures may be one-dimensional, two-dimensional, three-dimensional, and even four-dimensional if produced using an electromagnetic array.

Programmed magnetic structures may be developed from ferrites, rare-earth materials (e.g. Neodymium magnet, Samarium-cobalt magnet, neodymium iron boron), ceramics, and electromagnets alike, and the correlation effects may he scalable from very large permanent magnets to nanometer-scale devices. Multipole magnetic devices may be constructed from discrete permanent magnets, or by exposing heated magnetizable material to a coded magnetic field.

The programmed magnets may then be formed together with the pylon (406). The extremity unit may also be formed with the programmed magnets (408).

Pylons that may be made of titanium or aluminum may be die-cast; in this process, liquid metal may be forced into a steel die of the proper shape. The wooden pieces may be planed, sawed, and drilled. The various components may be put together in a variety of ways, using bolts, adhesives, and laminating, to name a few.

The entire limb may be assembled by the prosthetist's technician using such tools as a torque wrench and screwdriver to bolt the prosthesis device together (410). After this, the prosthetist again fits the permanent socket to the patient, this time with the completed custom-made limb attached. Final adjustments may be then made.

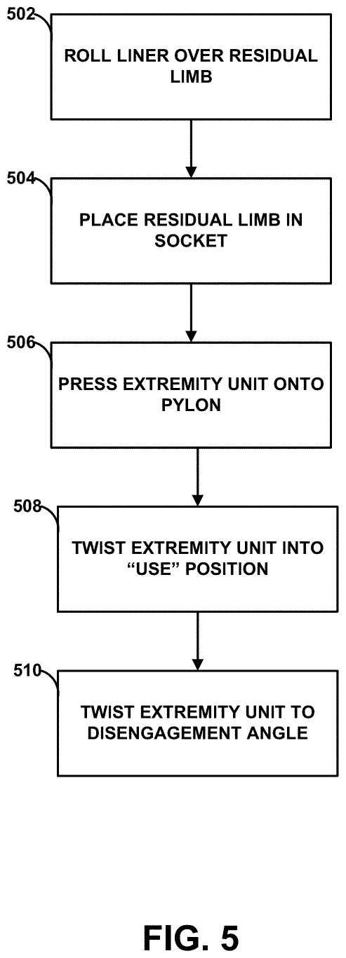

FIG. 5 is a flow diagram of a method of use for a prosthesis system in accordance with examples of the present disclosure. The wearer may begin by rolling a liner on to the residual limb (502). The wearer may unroll the liner so it's inside out. Then, place the stump into the bottom of the liner. The liner may be rolled back up over the residual limb. It should fit snugly, but not feel uncomfortable. When the liner is fully in place, a strap or pin emerging from the bottom of the liner should be centered over the residual limb.

The residual limb may then be placed within a socket on the socket housing (504). An extremity unit may be turned to a detachment angle to align the second interlock structure with the first interlock structure. The wearer may then push the extremity unit onto the pylon (506). This may take a little bit of pressure as the detachment angle is also the maximum magnetic repulsion of the first and second interlocking programmed magnets. The wearer may make a quick twist of the extremity unit towards an alignment angle (e.g., the use position) (508).

The wearer may feel a magnetic force as the extremity unit moves closer to the state of maximal magnetic attraction. The wearer may then use the prosthesis system to live their everyday life or perhaps use the extremity unit for a specific purpose, such as running, climbing or walking. When the wearer is finished with the particular activity and wishes to remove the extremity unit, they may simply sit and twist the extremity unit toward the disengagement angle and pull the extremity unit off of the pylon (510).

The following is a non-limiting list of examples that are in accordance with one or more techniques of this disclosure.

### EXAMPLE 1A

A prosthesis system comprising a socket housing comprising a socket configured to receive a residual limb of a wearer; a pylon extending distally of the socket; a first interlock structure at a distal end of the pylon, wherein the first interlock structure comprises a first one or more programmed magnets; and an extremity unit configured to be attached to and removed from the pylon, wherein the extremity unit comprises a second interlock structure comprising a second one or more programmed magnets; wherein the pylon and the extremity unit are configured such that, when the extremity unit is attached to the pylon, the first and second one or more programmed magnets are positioned and oriented to magnetically attract each other and secure the extremity unit to the pylon.

### EXAMPLE 2A

The prosthesis system of example 1A, wherein the first and second one or more programmed magnets are configured such that movement of the extremity unit in a predetermined direction relative to the pylon releases the magnetic attraction of the first and second one or more programmed magnets to each other.

### EXAMPLE 3A

The prosthesis system of any one of examples 1A or 2A, wherein the first and second one or more programmed magnets comprise twist release programmed magnets, and the predetermined direction comprises rotation of the extremity unit about a longitudinal axis of the pylon.

### EXAMPLE 4A

The prosthesis system of any one of examples 1A, 2A, or 3A, wherein the first interlock structure comprises a cylinder extending longitudinally away from the pylon, the cylinder with at least one cam extending outward transversely from the longitudinal axis.

### EXAMPLE 5A

The prosthesis system of any one of examples 1A, 2A, or 3A, wherein the first one or more programmed magnets and the second one or more programmed magnets are programed to have a greater magnetic repel force when the first one or more programmed magnets and the second one or more programmed magnets are positioned so the pylon and foot have been rotated about the longitudinal axis out of proper alignment by at least 90 degrees.

### EXAMPLE 6A

The prosthesis system of any one of examples 1A, 2A, or 3A, wherein the first one or more programmed magnets and the second one or more programmed magnets are programed to have a greater magnetic repel force when the first one or more programmed magnet and the second one or more programmed magnet are positioned so the pylon and foot have been rotated about the longitudinal axis out of proper alignment by at least 45 degrees.

### EXAMPLE 7A

The prosthesis system of example 1A, wherein the extremity unit comprises a prosthesis foot.

### EXAMPLE 8A

The prosthesis system of example 1A, wherein the socket is configured to receive a residual limb.

### EXAMPLE 9A

The prosthesis system of example 1A, wherein the first one or more programmed magnets and the second one or more programmed magnets are programed to have a greater magnetic pull force when the first one or more programmed magnets and the second one or more programmed magnets are positioned so the pylon and foot are properly aligned for use by a wearer.

### EXAMPLE 10A

The prosthesis system of example 1A, wherein the first interlock structure comprises a three-dimensional key comprising a cylindrical portion extending longitudinally from the socket housing, and the second interlock structure comprises a cylindrical channel configured to receive the cylindrical portion.

### EXAMPLE 11A

The prosthesis system of example 10A, wherein the cylindrical portion comprises at least one cam portion extending outward from the cylindrical portion, and the cylindrical channel comprises at least one slot that is configured to receive the at least one cam portion.

### EXAMPLE 1B

A method of manufacturing a prosthesis system, the method comprising forming a socket housing configured to receive a residual limb of a wearer, the socket housing comprising: a socket configured to receive a residual limb of a wearer; a pylon extending distally of the socket; and a first interlock structure at a distal end of the pylon, wherein the first interlock structure comprises a first one or more programmed magnets; and forming an extremity unit configured to be attached to and removed from the pylon, wherein the extremity unit comprises a second interlock structure comprising a second one or more programmed magnets, wherein forming the socket housing and the extremity unit comprises positioning and orienting the first and second one or more programmed magnets such that, when the extremity unit is attached to the pylon, the first and second one or more programmed magnets magnetically attract each other and secure the extremity unit to the pylon.

### EXAMPLE 2B

The method of example 1B, further comprising programming the first and second one or more programmed magnets such that movement of the extremity unit in a predetermined direction relative to the pylon releases the magnetic attraction of the first and second plurality of programmed magnets to each other.

### EXAMPLE 3B

The method of example 2B, wherein the programming further comprises programming the first one or more programmed magnets and the second one or more programmed magnets to have a greater magnetic repel force when the first one or more programmed magnet and the second one or more programmed magnet are positioned so the pylon and a foot have been rotated about a longitudinal axis out of proper alignment by at least 90 degrees.

### EXAMPLE 4B

The method of any one of examples 2B and 3B, wherein the programming further comprises programming the first one or more programmed magnets and the second one or more programmed magnets to have a greater magnetic repel force when the first one or more programmed magnet and the second one or more programmed magnet are positioned so the pylon and an extremity unit have been rotated about the longitudinal axis out of proper alignment by at least 45 degrees.

### EXAMPLE 5B

The method of example 2B, wherein the first and second one or more programmed magnets comprise twist and release programmed magnets and the predetermined direction comprises rotation of the extremity unit about a longitudinal axis of the pylon.

### EXAMPLE 6B

The method of any one of examples 2B or 5B, wherein the first interlock structure comprises a cylinder extending longitudinally away from the pylon, the cylinder with at least one cam extending outward transversely from the longitudinal axis.

### EXAMPLE 7B

The method of example 1B, further comprising printing three-dimensionally the first and the second interlock structures with respective magnetic structures comprising the one or more first and second programmed magnets.

### EXAMPLE 8B

The method of example 1B, wherein the extremity unit comprises a prosthesis foot.

### EXAMPLE 9B

The method of example 1B, further comprising configuring the socket to receive a residual limb.

### EXAMPLE 10B

The method of example 1B, further comprising programming the first one or more programmed magnets and the second one or more programmed magnets to have a greater magnetic pull force when the first one or more programmed magnet and the second one or more programmed magnet are positioned so the pylon and a foot are properly aligned for use by a wearer.

### EXAMPLE 11B

The method of example 1B, wherein the first interlock structure comprises a three-dimensional key comprising a cylindrical portion extending longitudinally from the socket housing, and the second interlock structure comprises a cylindrical channel configured to receive the cylindrical portion.

### EXAMPLE 12B

The method of example 11B, wherein the cylindrical portion has at least one cam portion extending outward from the cylindrical portion, and the cylindrical channel comprises at least one slot that is configured to receive the at least one cam portion.

### Example 1C

A prosthesis system comprising: a socket housing comprising; a socket configured to receive a residual limb of a wearer; a pylon extending distally of the socket; a first interlock structure at a distal end of the pylon, the first interlock structure comprising: one or more programmed magnets; a three-dimensional key comprising a cylinder extending longitudinally away from the pylon, the cylinder with at least one cam extending outward transversely from the longitudinal axis; and an extremity unit configured to be attached to and removed from the pylon, wherein the extremity unit comprises: a second interlock structure comprising one or more programmed magnets, the second interlock comprising a cylindrical channel that comprises at least one slot that is configured to receive the at least one cam portion; wherein the pylon and the extremity unit are configured such that, when the extremity unit is attached to the pylon, the one or more programmed magnets of the first interlock structure and the second interlock structure are positioned and oriented to magnetically attract each other and secure the extremity unit to the pylon; wherein the first and second one or more programmed magnets are configured such that movement of the extremity unit in a predetermined direction relative to the pylon releases the magnetic attraction of the first and second one or more programmed magnets to each other and the first and second one or more programmed magnets comprise twist release programmed magnets, and the predetermined direction comprises rotation of the extremity unit about a longitudinal axis of the pylon; wherein the first one or more programmed magnets and the second one or more programmed magnets are programed to have a greater magnetic pull force when the first one or more programmed magnets and the second one or more programmed magnets are positioned so the pylon and foot are properly aligned for use by a wearer.

Various examples have been described herein. Any combination of the described operations or functions may be contemplated. These and other examples may be within the scope of the following claims.

What is claimed is:

1. A prosthesis system comprising:
a socket housing comprising;
a socket configured to receive a residual limb of a wearer;
a pylon extending distally of the socket;
a first interlock structure at a distal end of the pylon, wherein the first interlock structure comprises a first one or more programmed magnets; and
an extremity unit configured to be attached to and removed from the pylon, wherein the extremity unit comprises a second interlock structure comprising a second one or more programmed magnets;
wherein the pylon and the extremity unit are configured such that, when the extremity unit is attached to the pylon, the first and second one or more programmed magnets are positioned and oriented to magnetically attract each other and secure the extremity unit to the pylon.

2. The prosthesis system of claim 1, wherein the first and second one or more programmed magnets are configured such that movement of the extremity unit in a predetermined direction relative to the pylon releases the magnetic attraction of the first and second one or more programmed magnets to each other.

3. The prosthesis system of claim 2, wherein the first and second one or more programmed magnets comprise twist release programmed magnets, and the predetermined direction comprises rotation of the extremity unit about a longitudinal axis of the pylon.

4. The prosthesis system of claim 3, wherein the first interlock structure comprises a cylinder extending longitudinally away from the pylon, the cylinder with at least one cam extending outward transversely from the longitudinal axis.

5. The prosthesis system of claim 3, wherein the first one or more programmed magnets and the second one or more programmed magnets are programed to have a greater magnetic repel force when the first one or more programmed magnets and the second one or more programmed magnets are positioned so the pylon and foot have been rotated about the longitudinal axis out of proper alignment by at least 90 degrees.

6. The prosthesis system of claim 3, wherein the first one or more programmed magnets and the second one or more programmed magnets are programed to have a greater magnetic repel force when the first one or more programmed magnet and the second one or more programmed magnet are positioned so the pylon and foot have been rotated about the longitudinal axis out of proper alignment by at least 45 degrees.

7. The prosthesis system of claim 1, wherein the extremity unit comprises a prosthesis foot.

8. The prosthesis system of claim 1, wherein the socket is configured to receive a residual limb.

9. The prosthesis system of claim 1, wherein the first one or more programmed magnets and the second one or more programmed magnets are programed to have a greater magnetic pull force when the first one or more programmed magnets and the second one or more programmed magnets are positioned so the pylon and foot are properly aligned for use by a wearer.

10. The prosthesis system of claim 1, wherein the first interlock structure comprises a three-dimensional key comprising a cylindrical portion extending longitudinally from the socket housing, and the second interlock structure comprises a cylindrical channel configured to receive the cylindrical portion.

11. The prosthesis system of claim 10, wherein the cylindrical portion comprises at least one cam portion extending outward from the cylindrical portion, and the cylindrical channel comprises at least one slot that is configured to receive the at least one cam portion.

12. A method of manufacturing a prosthesis system, the method comprising:
forming a socket housing configured to receive a residual limb of a wearer, the socket housing comprising:
a socket configured to receive a residual limb of a wearer;
a pylon extending distally of the socket; and
a first interlock structure at a distal end of the pylon, wherein the first
interlock structure comprises a first one or more programmed magnets; and
forming an extremity unit configured to be attached to and removed from the pylon, wherein the extremity unit comprises a second interlock structure comprising a second one or more programmed magnets,
wherein forming the socket housing and the extremity unit comprises positioning and orienting the first and second one or more programmed magnets such that, when the extremity unit is attached to the pylon, the first and second one or more programmed magnets magnetically attract each other and secure the extremity unit to the pylon.

13. The method of claim 12, further comprising programming the first and second one or more programmed magnets such that movement of the extremity unit in a predetermined direction relative to the pylon releases the magnetic attraction of the first and second plurality of programmed magnets to each other.

14. The method of claim 13, wherein the programming further comprises programming the first one or more programmed magnets and the second one or more programmed magnets to have a greater magnetic repel force when the first one or more programmed magnet and the second one or more programmed magnet are positioned so the pylon and a foot have been rotated about a longitudinal axis out of proper alignment by at least 90 degrees.

15. The method of claim 13, wherein the programming further comprises programming the first one or more programmed magnets and the second one or more programmed magnets to have a greater magnetic repel force when the first one or more programmed magnet and the second one or more programmed magnet are positioned so the pylon and an extremity unit have been rotated about the longitudinal axis out of proper alignment by at least 45 degrees.

16. The method of claim 13, wherein the first and second one or more programmed magnets comprise twist and release programmed magnets and the predetermined direction comprises rotation of the extremity unit about a longitudinal axis of the pylon.

17. The method of claim 16, wherein the first interlock structure comprises a cylinder extending longitudinally away from the pylon, the cylinder with at least one cam extending outward transversely from the longitudinal axis.

18. The method of claim 12, further comprising printing three-dimensionally the first and the second interlock structures with respective magnetic structures comprising the one or more first and second programmed magnets.

19. The method of claim 12, wherein the extremity unit comprises a prosthesis foot.

20. The method of claim 12, further comprising configuring the socket to receive a residual limb.

21. The method of claim 12, further comprising programming the first one or more programmed magnets and the second one or more programmed magnets to have a greater magnetic pull force when the first one or more programmed magnet and the second one or more programmed magnet are positioned so the pylon and a foot are properly aligned for use by a wearer.

22. The method of claim 12, wherein the first interlock structure comprises a three-dimensional key comprising a cylindrical portion extending longitudinally from the socket housing, and the second interlock structure comprises a cylindrical channel configured to receive the cylindrical portion.

23. The method of claim 22, wherein the cylindrical portion has at least one cam portion extending outward from the cylindrical portion, and the cylindrical channel comprises at least one slot that is configured to receive the at least one cam portion.

24. A prosthesis system comprising:
a socket housing comprising;
a socket configured to receive a residual limb of a wearer;
a pylon extending distally of the socket;
a first interlock structure at a distal end of the pylon, the first interlock structure comprising:
one or more programmed magnets;
a three-dimensional key comprising a cylinder extending longitudinally away from the pylon, the cylinder with at least one cam
extending outward transversely from the longitudinal axis; and
an extremity unit configured to be attached to and removed from the pylon, wherein the extremity unit comprises:
a second interlock structure comprising one or more programmed magnets, the second interlock comprising a cylindrical channel that comprises at least one slot that is configured to receive the at least one cam portion;
wherein the pylon and the extremity unit are configured such that, when the extremity unit is attached to the pylon, the one or more programmed magnets of the first interlock structure and the second interlock structure are positioned and oriented to magnetically attract each other and secure the extremity unit to the pylon;
wherein the first and second one or more programmed magnets are configured such that movement of the extremity unit in a predetermined direction relative to the pylon releases the magnetic attraction of the first and second one or more programmed magnets to each other and the first and second one or more programmed magnets comprise twist release programmed magnets, and the predetermined direction comprises rotation of the extremity unit about a longitudinal axis of the pylon;
wherein the first one or more programmed magnets and the second one or more programmed magnets are programed to have a greater magnetic pull force when the first one or more programmed magnets and the second one or more programmed magnets are positioned so the pylon and foot are properly aligned for use by a wearer.
